(19) United States
(12) Patent Application Publication
(10) Pub. No.: US 2021/0341717 A1
(43) Pub. Date: Nov. 4, 2021

(54) VAPOR CELL AND VAPOR CELL MANUFACTURING METHOD

(71) Applicants: Kabushiki Kaisha US Research, Sendai-shi (JP); National Institute of Information and Communications Technology, Tokyo (JP); Tamagawa Holdings Co., Ltd., Tokyo (JP); Synergetics Co., Ltd., Tokyo (JP)

(72) Inventors: Takahito Ono, Sendai-shi (JP); Hitoshi Nishino, Sendai-shi (JP); Masaya Toda, Sendai-shi (JP); Motoaki Hara, Tokyo (JP); Yuichiro Yano, Tokyo (JP)

(21) Appl. No.: 17/283,091

(22) PCT Filed: Oct. 9, 2019
(86) PCT No.: PCT/JP2019/039773

§ 371 Date: Apr. 6, 2021

(30) Foreign Application Priority Data
Oct. 10, 2018 (JP) 2018-191550

(51) Int. Cl. G02B 17/00; H03L 7/26

(52) U.S. Cl. CPC G02B 17/004; A61B 5/245; H03L 7/26

(57) ABSTRACT

A vapor cell which can increase the S/N ratio of light as a signal and has high accuracy and a vapor cell manufacturing method are provided. The vapor cell includes: a reflection space (14) provided so as to be able to store a gas containing an alkali metal atom; and an incident light reflection surface, an in-plane reflection portion (17), and an emission light reflection surface provided inside the reflection space (14). The incident light reflection surface has an elevation angle of 45° from an optical path plane so that the incident light incident from a predetermined external direction is reflected in the optical path plane that is perpendicular to the incident light. The in-plane reflection portion (17) has a reflection surface that reflects the reflected light from the incident light reflection surface, the reflection surface being substantially perpendicular to the optical path plane so that the reflected light from the incident light reflection surface is reflected in the optical path plane once or multiple times. The emission light reflection surface has an elevation angle 45° from the optical path plane so that the reflected light from the in-plane reflection portion (17) is reflected in a direction substantially perpendicular to the optical path plane and an emission light is emitted to the outside.

### CROSS REFERENCE TO RELATED APPLICATIONS

This application is a U.S. National Phase Application of PCT International Application Number PCT/JP2019/039773, filed on Oct. 9, 2019, designating the United States of America and published in the Japanese language, which is an International Application of and claims the benefit of priority to Japanese Patent Application No. 2018-191550, filed on Oct. 10, 2018. The disclosures of the above-referenced applications are hereby expressly incorporated by reference in their entireties.

### FIELD OF THE INVENTION

The present invention relates to a vapor cell and a vapor cell manufacturing method.

### DESCRIPTION OF RELATED ART

Conventionally, as a device which uses a vapor cell in which an atom is sealed, a high-precision atomic clock based on the frequency of an electromagnetic wave absorbed by the atom (see, for example, Non-Patent Literature 1) and a magnetic sensor which uses optical pumping of the atom (see, for example, Patent Literature 1) have been developed. Further, in order to reduce the size of these devices, vapor cells are also manufactured by MEMS technology. However, when the size of vapor cells is reduced, there is a problem that the optical path length of a laser beam or the like incident on the vapor cell is shortened and the S/N ratio is lowered.

Therefore, in order to solve this problem, a reflection-type vapor cell that can extend the optical path length by reflecting the laser beam in the vapor cell in a direction parallel to the substrate surface of the vapor cell has been developed (for example, see Non-Patent Literature 2). Since this vapor cell can be formed thin, and an incident window and output window of the laser beam can be formed on the same surface of the vapor cell, the vapor cell can be easily mounted in a circuit.

Further, in this reflection-type vapor cell, the (111) plane is formed by crystal anisotropic wet etching using a silicon wafer cut on the (100) plane, and the (100) plane is used as a reflection surface. Since the (111) plane is at 54.74° with respect to the substrate surface of the silicon wafer, the incident light and the emission light are bent using a diffraction grating in order to reflect light incident perpendicularly to the substrate surface in a direction parallel to the substrate surface and emit the reflected light perpendicularly to the substrate surface.

A method in which a surface being at 45° with respect to the surface of a silicon wafer is manufactured by performing crystal anisotropic etching using a silicon wafer having an off angle of 9.74° from the (100) plane is known (see, for example, Non-Patent Literature 3).

### CITATION LIST

Patent Literature 1: Japanese Patent No. 5786546

Non-Patent Literature 1: M. Hara, et al., “Micro Atomic Frequency Standards Employing An Integrated FBAR-VCO Oscillating On The ⁸⁷RB Clock Frequency Without A Phase Locked Loop”, IEEE, MEMS 2018, p. 715-718

Non-Patent Literature 2: Ravinder Chutani et al, “Laser light routing in an elongated micromachined vapor cell with diffraction gratings for atomic clock applications”, Sci. Rep., 2015, 5, 14001

Non-Patent Literature 3: Carola Strandman et al, “Fabrication of 45° mirrors together with well-defined v-grooves using wet anisotropic etching of silicon”, IEEE J. Microelectromech. Syst., 1995, Vol. 4, No. 4, p. 213-219

### SUMMARY OF THE INVENTION

In the reflection-type vapor cell disclosed in Non-Patent Literature 2, there is a problem that, when light is diffracted by a diffraction grating, since the intensity of light is lowered, the S/N ratio of light as a signal is reduced and the accuracy is lowered.

The present invention has been formed in view of such problems, and an object of the present invention is to provide a vapor cell which can increase the S/N ratio of light as a signal and has high accuracy and to provide a vapor cell manufacturing method.

In order to attain the objective, a vapor cell according to the present invention includes: a reflection space provided so as to be able to store gas containing an alkali metal atom; and an incident light reflection surface, an in-plane reflection portion, and an emission light reflection surface provided inside the reflection space, wherein the incident light reflection surface has an elevation angle of approximately 45° from an optical path plane so that incident light incident from an external predetermined direction is reflected in the optical path plane that is substantially perpendicular to the incident light, the in-plane reflection portion has a reflection surface that reflects the reflected light from the incident light reflection surface, the reflection surface being substantially perpendicular to the optical path plane so that the reflected light from the incident light reflection surface is reflected in the optical path plane once or multiple times, and the emission light reflection surface has an elevation angle of approximately 45° from the optical path plane so that the reflected light from the in-plane reflection portion is reflected in a direction substantially perpendicular to the optical path plane and an emission light is emitted to the outside.

Since the vapor cell according to the present invention can utilize the incident light and the emission light forming directions substantially perpendicular to the optical path plane, it is easy to design and install the incident light irradiating means, the emission light receiving means, and the like, and it is not necessary to bend the incident light and the emission light with a diffraction grating or the like. Further, even in the reflection space, the light is only reflected by the reflection surface and is not diffracted, so that the decrease in the intensity of the light can be suppressed. Therefore, the S/N ratio of light as a signal can be increased, and high accuracy can be obtained. Further, in the vapor cell according to the present invention, since light passes through the optical path plane while being reflected by the in-plane reflection portion until the incident light is reflected by the incident/emission light reflection surface and the emission light is emitted outside after the incident light is reflected by the incident/emission light reflection surface to enter the optical path plane, the optical path length can be increased. As a result, the accuracy can be further improved.

Since the vapor cell according to the present invention allows light to pass through the optical path plane while being reflected by the in-plane reflection portion, the thickness in the direction perpendicular to the optical path plane can be reduced. Therefore, the installation space in a circuit or the like can be reduced. Further, the vapor cell according to the present invention is easy to design because the angles formed by the incident light reflection surface, the emission light reflection surface, the reflection surface of the incident light reflection surface, and the optical path plane are approximately 45° or 90°.

Although the number of reflections in the in-plane reflection portion is not particularly limited in the vapor cell according to the present invention, the larger number of reflections is preferable to increase the optical path length. Moreover, the alkali metal atom is not particularly limited, and for example, Cs or Rb is preferably used. Further, in order to further increase the accuracy, the reflection space is preferably sealed.

In the vapor cell according to the present invention, preferably, the emission light reflection surface is provided so as to emit the emission light in a direction parallel to and opposite to an incident direction of the incident light. In this case, the incident window of the incident light and the output window of the emission light can be manufactured on the same side of the vapor cell, and the vapor cell can be easily mounted on a circuit or the like.

In the vapor cell according to the present invention, the incident light reflection surface and the emission light reflection surface may be formed of the same one surface, and the in-plane reflection portion may be provided so that the reflected light reflected by the incident light reflection surface and the reflected light incident on the emission light reflection surface travel in opposite directions and in parallel to each other. In this case, the emission light can be emitted in a direction parallel to and opposite to the incident direction of the incident light. Moreover, in this case, preferably, the in-plane reflection portion has a first reflection surface provided to reflect the reflected light reflected by the incident light reflection surface and bend a traveling direction of the reflected light by 90° and a second reflection surface provided to reflect the reflected light reflected by the first reflection surface and bend a traveling direction of the reflected light by 90°.

In the vapor cell according to the present invention, the incident light reflection surface, the reflection surface of the in-plane reflection portion that reflects the reflected light from the incident light reflection surface, and the emission light reflection surface may be covered with a dielectric multilayer film or a metal film that does not react with the alkali metal atom. When the surface is covered with the dielectric multilayer film, the reflectance of each reflection surface can be increased. Further, when the surface is covered with the metal film, each reflection surface can be protected. The metal film is, for example, a Ti/Pt/Au film or a Ti/Au film whose surface is formed of a Ti layer.

The vapor cell according to the present invention preferably includes a storage space for storing an alkali metal dispenser capable of releasing the alkali metal atom, the storage space being provided such that air can pass between the storage space and the reflection space. In this case, the alkali metal atom released from the alkali metal dispenser stored in the storage space can be supplied to the inside of the reflection space. The reflection space and the storage space are preferably sealed.

A vapor cell manufacturing method according to the present invention is a vapor cell manufacturing method for manufacturing the vapor cell according to the present invention and includes: performing crystal anisotropic etching on a planar silicon to form the incident light reflection surface and the emission light reflection surface; and performing deep reactive ion etching (DRIE) on the silicon to form the reflection surface of the in-plane reflection portion that reflects reflected light from the incident light reflection surface.

The vapor cell manufacturing method according to the present invention can manufacture the vapor cell according to the present invention relatively easily and accurately. In the vapor cell manufacturing method according to the present invention, preferably, the silicon is formed of a silicon wafer having an off angle of 9.74° from the (100) plane. In this case, a plane that is at 45° with respect to the surface of the silicon wafer can be manufactured by crystal anisotropic etching. As a result, the optical path plane can be formed as a plane parallel to the surface of the silicon wafer and the incident light reflection surface and the emission light reflection surface can be formed with an elevation angle of 45° from the optical path plane.

In the vapor cell manufacturing method according to the present invention, preferably, hydrogen annealing is performed at a temperature of 1000° C. or higher after the crystal anisotropic etching and the deep reactive ion etching are performed. In this case, the surface flow of silicon is generated by a heat treatment process and the incident light reflection surface, the emission light reflection surface, and the reflection surface of the in-plane reflection portion formed by etching can be planarized.

In the vapor cell manufacturing method according to the present invention, after the crystal anisotropic etching and the deep reactive ion etching are performed, or after the hydrogen annealing is performed, a dielectric multilayer film or a metal film that does not react with the alkali metal atom may be formed by deposition on the incident light reflection surface, the emission light reflection surface, and the reflection surface of the in-plane reflection portion. Moreover, in this case, preferably, the deposition is performed so that a deposition material collides with the incident light reflection surface, the emission light reflection surface, and the reflection surface of the in-plane reflection portion at the same angle. In this way, the dielectric multilayer film or the metal film can be formed with substantially the same thickness at the same time on the respective reflection surfaces.

In the vapor cell manufacturing method according to the present invention, preferably, after the incident light reflection surface, the emission light reflection surface, and the reflection surface of the in-plane reflection portion are formed, or after the dielectric multilayer film is formed, the silicon is sandwiched between a pair of glass plates to seal the reflection space. When the storage space is provided, it is preferable to seal the storage space together with the reflection space. In this case, a vapor cell with a higher precision can be manufactured.

According to the present invention, it is possible to provide a vapor cell which can increase the S/N ratio of light as a signal and has high accuracy and to provide a vapor cell manufacturing method.

### BRIEF DESCRIPTION OF THE DRAWINGS

FIGS. 1A and 1B illustrate a vapor cell according to an embodiment of the present invention in which FIG. 1A is a plan view and FIG. 1B is a cross-sectional view along A-A′ in FIG. 1A.

FIGS. 2A to 2D are cross-sectional views illustrating a vapor cell manufacturing method according to an embodiment of the present invention.

FIGS. 3A to 3F illustrate a vapor cell manufacturing method according to an embodiment of the present invention, in which FIG. 3A is a plan view, FIGS. 3B and 3C are cross-sectional views along A-A′ in FIG. 3A, FIG. 3D is a bottom view, and FIGS. 3E and 3F are cross-sectional views along A-A′ in FIG. 3D.

FIGS. 4A to 4E illustrate a vapor cell manufacturing method according to an embodiment of the present invention, in which FIG. 4A is a plan view, FIGS. 4B and 4C are cross-sectional views along A-A′ in FIG. 4A, FIG. 4D is a plan view, and FIG. 4E is a cross-sectional view along A-A′ in FIG. 4D.

FIG. 5A is a cross-sectional view illustrating a modified example of the vapor cell according to the embodiment of the present invention, and FIG. 5B is a cross-sectional view illustrating a method of manufacturing the vapor cell illustrated in FIG. 5A.

FIGS. 6A to 6D illustrate a reflection space formed in a silicon wafer of the vapor cell according to the embodiment of the present invention, in which FIG. 6A is a plan view of a modified example in which the reflection space forms a pentagon, FIG. 6B is a plan view of a modified example in which the number of reflections at an in-plane reflection portion is three times, FIG. 6C is a plan view of a modified example when the reflection angles on first and second reflection surfaces slightly deviate from 90°, and FIG. 6D is an enlarged plan view of the second reflection surface in FIG. 6C.

FIG. 7 is an absorption spectrum of the D1 line of Rb, of the vapor cell according to the embodiment of the present invention.

FIG. 8 is a CPT spectrum of the vapor cell of the embodiment of the present invention.

### DETAILED DESCRIPTION OF THE INVENTION

Hereinafter, an embodiment of the present invention will be described with reference to the drawings.

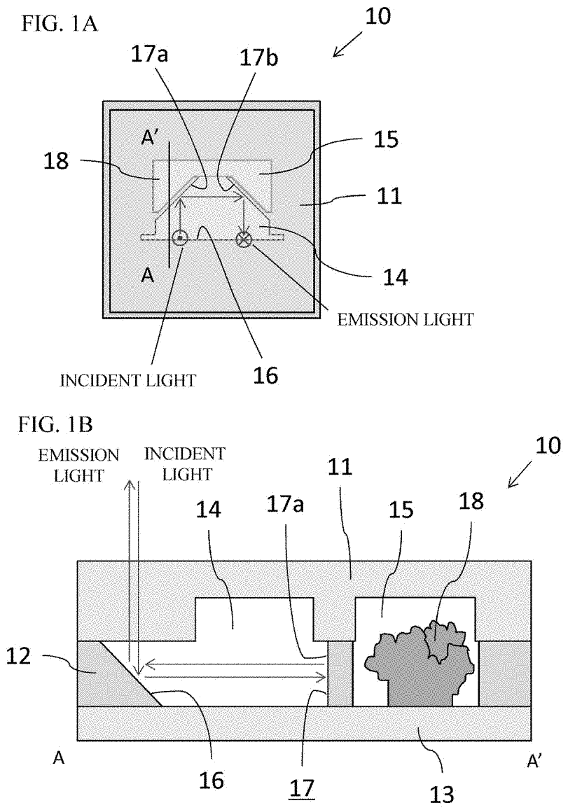

FIGS. 1 to 8 illustrate a vapor cell and a vapor cell manufacturing method according to an embodiment of the present invention.

As illustrated in FIGS. 1A and 1B, a vapor cell 10 has a three-layer structure including an upper glass plate 11, a silicon wafer 12, and a lower glass plate 13. In a specific example illustrated in FIGS. 1A and 1B, the upper glass plate 11 and the lower glass plate 13 are formed of Tempax glass. Further, the upper glass plate 11, the silicon wafer 12, and the lower glass plate 13 have thicknesses of 0.3 mm, 0.2 mm, and 1 mm, respectively.

The vapor cell 10 has a reflection space 14 and a storage space 15 between the upper glass plate 11 and the lower glass plate 13, the spaces being formed by processing the upper glass plate 11, the silicon wafer 12, and the lower glass plate 13. Further, the vapor cell 10 has an incident/emission light reflection surface 16 and an in-plane reflection portion 17 provided inside the reflection space 14, and has an alkali metal dispenser 18 inside the storage space 15.

As illustrated in FIG. 1B, the reflection space 14 and the storage space 15 are formed so as to penetrate the silicon wafer 12. The reflection space 14 and the storage space 15 are arranged side by side along the surface of the silicon wafer 12 and are provided so that air can pass between the reflection space 14 and the storage space 15. Further, the reflection space 14 and the storage space 15 are sealed with respect to the outside of the vapor cell 10. As illustrated in FIG. 1A, the reflection space 14 and the storage space 15 have a rectangular outer shape in a plan view, the reflection space 14 is on one long side, and the storage space 15 is on the other long side. In the reflection space 14, the boundary line with respect to the storage space 15 in a plan view protrudes in a mountain shape on the storage space 15 side, the apex of the mountain shape is parallel to the long side, and the mountain skirt portion is at 45° with respect to the long side.

As illustrated in FIGS. 1A and 1B, the incident/emission light reflection surface 16 forms one long side of the reflection space 14, has an angle of 45° with respect to the surface of the silicon wafer 12, and is provided in a state of being directed toward the inner side of the reflection space 14 and the upper glass plate 11. The in-plane reflection portion 17 has a first reflection surface 17a forming one mountain skirt portion in a plan view and a second reflection surface 17b forming the other mountain skirt portion in a plan view. The first reflection surface 17a and the second reflection surface 17b are provided in a state of forming an angle of 90° with respect to the surface of the silicon wafer 12 and being directed toward the inner side of the reflection space 14. The incident/emission light reflection surface 16 forms an incident light reflection surface and an emission light reflection surface.

The alkali metal dispenser 18 can release an alkali metal atom by heating and is provided inside the storage space 15. The alkali metal dispenser 18 may be any dispenser of a Cs dispenser or an Rb dispenser as long as it releases an alkali metal atom. In a specific example illustrated in FIGS. 1A and 1B, the alkali metal dispenser 18 is formed of an Rb dispenser. The vapor cell 10 is adapted to seal the gas containing an alkali metal atom (Rb) inside the storage space 15 and the reflection space 14 by the alkali metal atom released from the alkali metal dispenser 18.

As illustrated in FIGS. 1A and 1B, the vapor cell 10 is provided such that incident light incident in the direction perpendicular to the surface of the silicon wafer 12 from above the upper glass plate 11 is bent at 90° by being reflected by the incident/emission light reflection surface 16 and enters the optical path plane parallel to the surface of the silicon wafer 12 to be directed toward the first reflection surface 17a of the in-plane reflection portion 17. Further, the vapor cell 10 is provided such that the reflected light of the incident light from the incident/emission light reflection surface 16 is bent at 90° in the optical path plane by being reflected by the first reflection surface 17a of the in-plane reflection portion 17 and is directed toward the second reflection surface 17b of the in-plane reflection portion 17. Furthermore, the vapor cell 10 is provided such that the reflected light from the first reflection surface 17a is bent at 90° in the optical path plane by being reflected by the second reflection surface 17b of the in-plane reflection portion 17 and is directed toward the incident/emission light reflection surface 16. As a result, in the vapor cell 10, the reflected light of the incident light reflected by the incident/emission light reflection surface 16 and the reflected light from the second reflection surface 17b incident on the incident/emission light reflection surface 16 travel in opposite directions in parallel to each other. Furthermore, the vapor cell 10 is provided such that the reflected light from the second reflection surface 17b is bent at 90° by being reflected by the incident/emission light reflection surface 16 to be directed toward the outside from the upper glass plate 11 and an emission light is emitted in a direction perpendicular to the surface of the silicon wafer 12. As a result, the vapor cell 10 emits the emission light in a direction parallel to and opposite to the incident direction of the incident light. The incident/emission light reflection surface 16 has an elevation angle of 45° from the optical path plane, and the first reflection surface 17a and the second reflection surface 17b of the in-plane reflection portion 17 are perpendicular to the optical path plane. In a specific example illustrated in FIGS. 1A and 1B, the optical path length inside the reflection space 14 is approximately 15 mm.

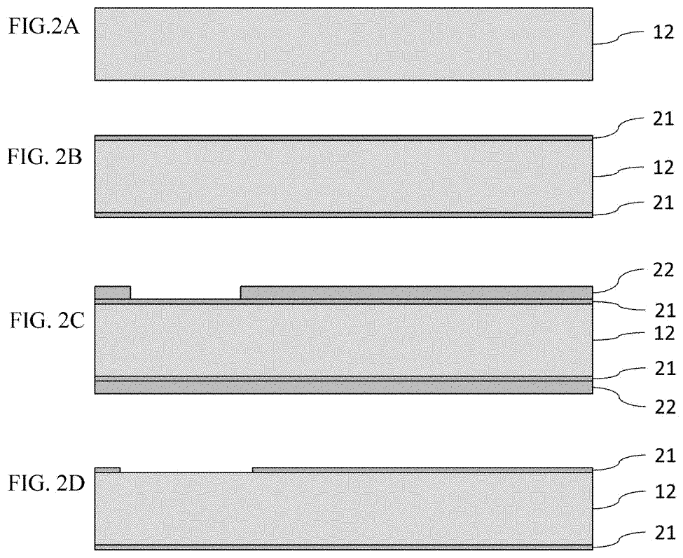

The vapor cell 10 is suitably manufactured by a vapor cell manufacturing method according to the embodiment of the present invention. That is, as illustrated in FIGS. 2A to 2D, in the vapor cell manufacturing method according to the embodiment of the present invention, first, a silicon wafer 12 having a thickness of 200 μm and an off angle of 9.74° from the (100) plane is used (see FIG. 2A), and the silicon wafer 12 is thermally oxidized to form a 500 nm SiO₂ film 21 on both surfaces (see FIG. 2B). Subsequently, a resist film 22 is patterned on both surfaces thereof (see FIG. 2C), and the SiO₂ film 21 at the position corresponding to the reflection space is etched with BHF (ultra-high purity buffered hydrofluoric acid) to remove the resist film 22 (see FIG. 2D).

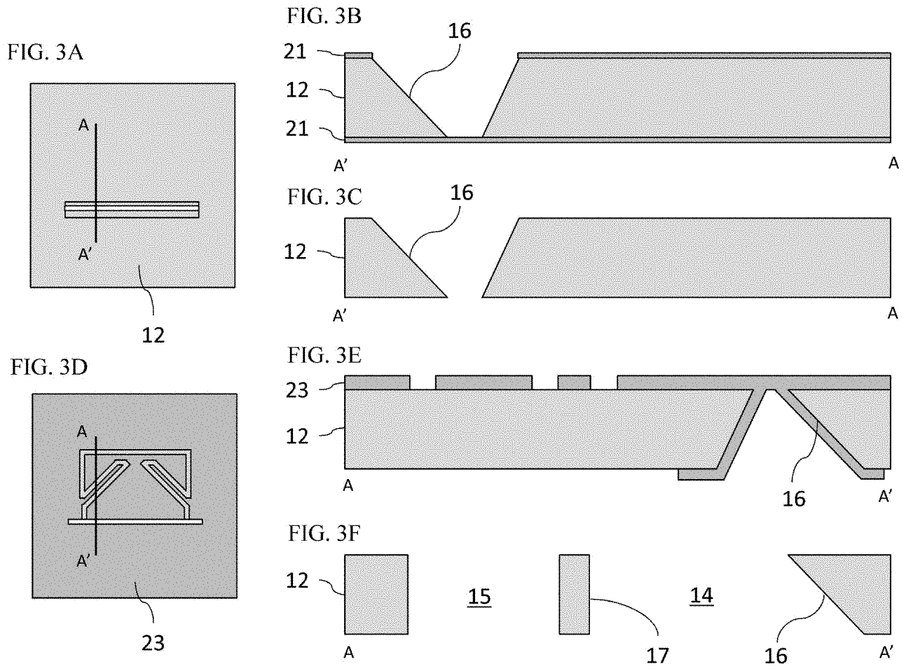

Subsequently, crystal anisotropic etching of Si is performed on a portion where Si is exposed using an aqueous potassium hydroxide solution (KOH) (see FIG. 3B). As a result, the incident/emission light reflection surface 16 forming 45° with respect to the surface of the silicon wafer 12 can be formed. Subsequently, the SiO₂ film 21 is completely etched and removed using BHF (see FIG. 3C). A resist film 23 is patterned on the surface of the exposed silicon wafer 12 (see FIG. 3E) and deep reactive ion etching (DRIE) is performed (see FIG. 3F). As a result, the first reflection surface 17a and the second reflection surface 17b of the in-plane reflection portion 17, the inner wall of the storage space 15, and the like can be formed, and the reflection space 14 and the storage space 15 can be formed.

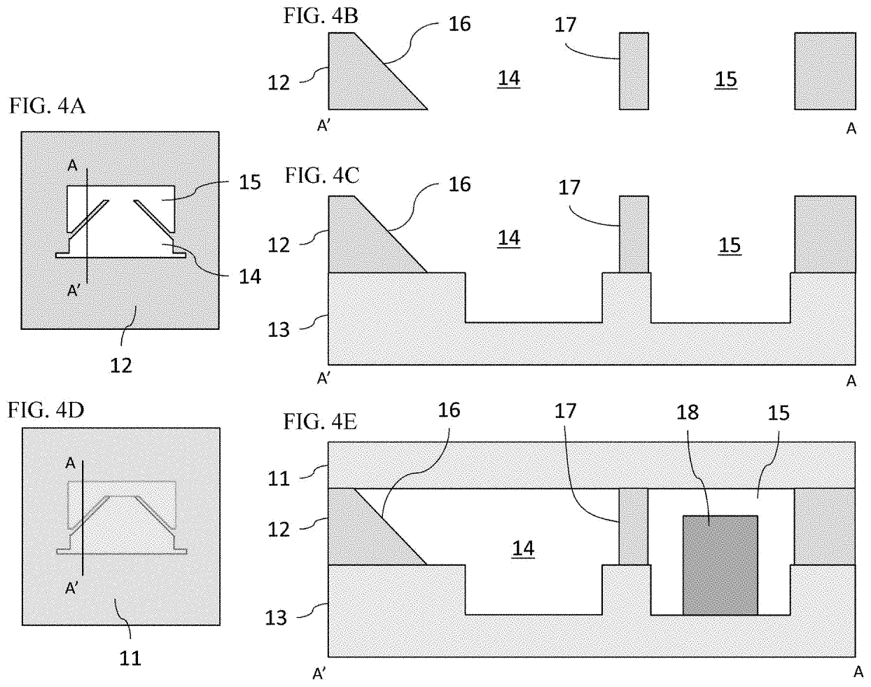

After the deep reactive ion etching, hydrogen annealing is performed at 1100° C. for 30 minutes (see FIG. 4B). As a result, the surface flow of silicon is generated, and the surfaces formed by each etching such as the incident/emission light reflection surface 16, the first reflection surface 17a and the second reflection surface 17b of the in-plane reflection portion 17 can be planarized. Subsequently, the lower glass plate 13 formed of Tempax glass having a thickness of 1 μm, in which recesses are formed at positions corresponding to the reflection space 14 and the storage space 15 using the patterning of a film resist and sandblasting, is anodic-bonded to one surface of the silicon wafer 12 (see FIG. 4B), and the alkali metal dispenser 18 is stored in the storage space 15. After that, another upper glass plate 11 formed of Tempax glass is anodic-bonded to the other surface of the silicon wafer 12 (see FIG. 4C). As a result, the silicon wafer 12 can be sandwiched between the upper glass plate 11 and the lower glass plate 13, and the reflection space 14 and the storage space 15 can be sealed. After sealing, the alkali metal dispenser 18 is activated with YAG laser light to generate Rb. As illustrated in FIGS. 1A and 1B, the upper glass plate 11 and the lower glass plate 13 may be bonded to the opposite surfaces of the silicon wafer 12, respectively. In this way, the vapor cell 10 can be manufactured relatively easily and accurately by the vapor cell manufacturing method according to the embodiment of the present invention.

Since the vapor cell 10 can utilize the incident light and the emission light forming directions substantially perpendicular to the optical path plane, it is easy to design and install the incident light irradiating means, the emission light receiving means, and the like, and it is not necessary to bend the incident light and the emission light with a diffraction grating or the like. Further, even in the reflection space, the light is only reflected by the reflection surface and is not diffracted, so that the decrease in the intensity of the light can be suppressed. Therefore, the S/N ratio of light as a signal can be increased, and high accuracy can be obtained. Further, in the vapor cell 10, since light passes through the optical path plane while being reflected by the in-plane reflection portion 17 until the incident light is reflected by the incident/emission light reflection surface 16 and the emission light is emitted outside after the incident light is reflected by the incident/emission light reflection surface 16 to enter the optical path plane, the optical path length can be increased. As a result, the accuracy can be further improved.

The vapor cell 10 is easy to design because the angles formed by the incident/emission light reflection surface 16, the first reflection surface 17a and the second reflection surface 17b of the in-plane reflection portion 17, and the optical path plane are 45° or 90°. Since the vapor cell 10 allows light to pass through the optical path plane while being reflected by the in-plane reflection portion 17, the thickness in the direction perpendicular to the optical path plane can be reduced. Therefore, the installation space in a circuit or the like can be reduced. Further, since the vapor cell 10 can emit the emission light in a direction parallel to and opposite to the incident direction of the incident light, the incident window of the incident light and the output window of the emission light can be manufactured on the same side of the vapor cell 10, and the vapor cell 10 can be easily mounted on a circuit or the like.

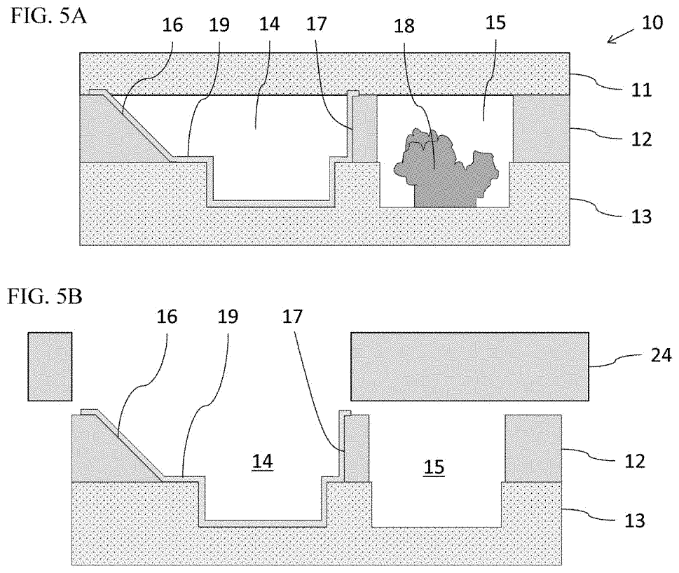

As illustrated in FIG. 5A, at least the incident/emission light reflection surface 16, the first reflection surface 17a and the second reflection surface 17b of the in-plane reflection portion 17 of the vapor cell 10 may be covered with a dielectric multilayer film 19. The dielectric multilayer film 19 is, for example, an Al₂O₃ film having a thickness of 20 nm. In this case, the dielectric multilayer film 19 can increase the reflectance of the incident/emission light reflection surface 16, the first reflection surface 17a and the second reflection surface 17b of the in-plane reflection portion 17.

For example, as illustrated in FIG. 5B, the dielectric multilayer film 19 can be formed by deposition, ALD (Atomic Layer Deposition), or the like after covering a portion of the surface of the storage space 15 or the silicon wafer 12 that does not form the dielectric multilayer film 19 with a stencil mask 24 after FIG. 4C. Further, when deposition or ALD is performed, it is preferable that the silicon wafer 12 and the lower glass plate 13 are relatively tilted with respect to the moving direction of the material of the dielectric multilayer film 19 so that the material of the dielectric multilayer film 19 collides with the incident/emission light reflection surface 16, the first reflection surface 17a and the second reflection surface 17b of the in-plane reflection portion 17 at the same angle. In the example illustrated in FIG. 5B, the silicon wafer 12 and the lower glass plate 13 are relatively tilted with respect to the moving direction of the material of the dielectric multilayer film 19 so that the angle formed by the moving direction of the material of the dielectric multilayer film 19 and the incident/emission light reflection surface 16 about the axis along the line of intersection between the incident/emission light reflection surface 16 and the optical path plane is 71.5°. As a result, since the angle between the moving direction of the material of the dielectric multilayer film 19 and the first reflection surface 17a and the second reflection surface 17b of the in-plane reflection portion 17 becomes 71.5°, the dielectric multilayer film 19 can be formed with substantially the same thickness at the same time on the respective reflection surfaces.

Instead of the dielectric multilayer film 19, a metal film that does not react with the alkali metal atom released by the alkali metal dispenser 18 may be provided. The metal film is, for example, a Ti/Pt/Au film or a Ti/Au film whose surface is formed of a Ti layer. The thickness of the Ti/Pt/Au film is, for example, 40/60/100 nm. The thickness of the Ti/Au film is, for example, 20/100 nm. In this case, the incident/emission light reflection surface 16, the first reflection surface 17a and the second reflection surface 17b of the in-plane reflection portion 17 can be protected.

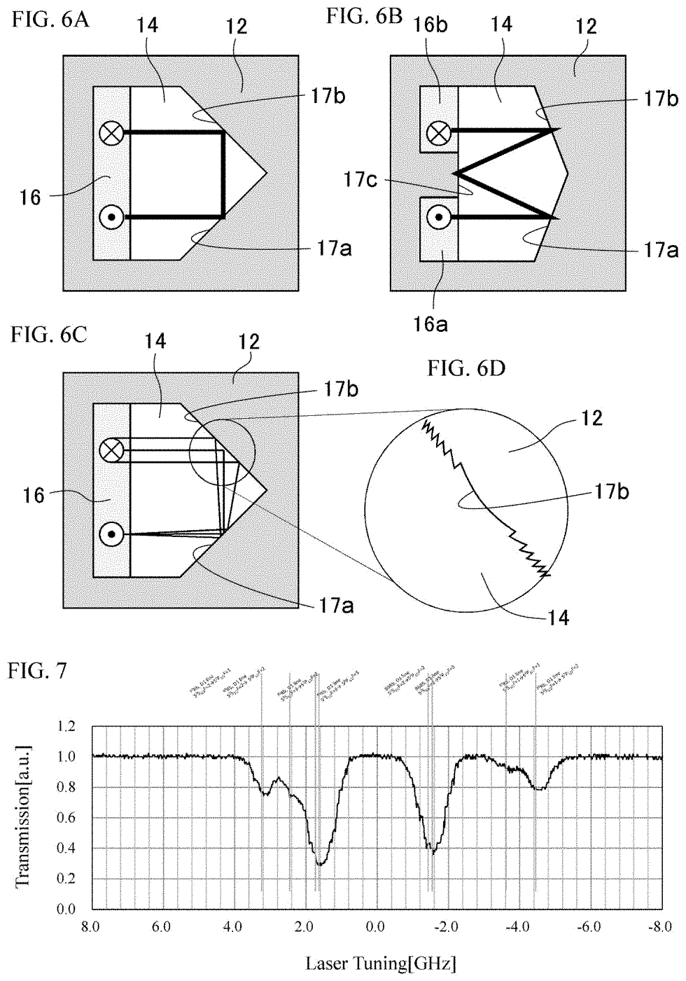

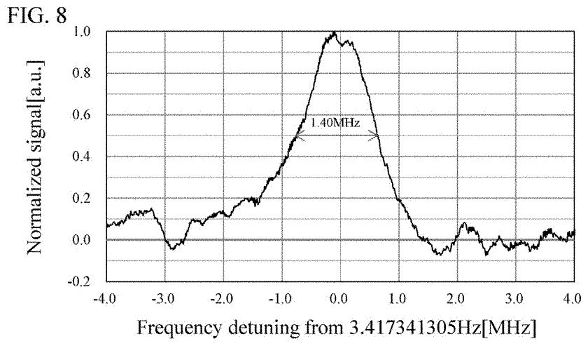

Further, as illustrated in FIG. 6A, in the vapor cell 10, the first reflection surface 17a and the second reflection surface 17b may be in contact with each other, and the reflection space 14 may form a pentagon in a plan view. Further, as illustrated in FIG. 6B, the vapor cell 10 may be provided such that the incident/emission light reflection surface 16 is divided into an incident light reflection surface 16a and an emission light reflection surface 16b, the in-plane reflection portion 17 has a third reflection surface 17c having an angle of 90° with respect to the surface of the silicon wafer 12 between the incident light reflection surface 16a and the emission light reflection surface 16b, and the reflection light entering the optical path plane from the incident light reflection surface 16a is reflected at an acute angle from the first reflection surface 17a, the third reflection surface 17c, and the second reflection surface 17b in that order and is directed toward the emission light reflection surface 16b. In this case, the number of reflections in the in-plane reflection unit 17 is three times, and the optical path length can be increased.

Further, as illustrated in FIG. 6C, in the vapor cell 10, the reflection angle on the first reflection surface 17a and the second reflection surface 17b is substantially 90°, and may slightly deviate from 90° rather than exactly 90° as illustrated in FIG. 1A and 1B. As illustrated in FIG. 6D, the first reflection surface 17a and the second reflection surface 17b may be curved or slightly tilted in the optical path plane due to deep reactive ion etching (DRIE) or the like. However, even in that case, the emission light from the incident/emission light reflection surface 16 can be emitted in a direction perpendicular to the surface of the silicon wafer 12, that is, in a direction parallel to and opposite to the incident direction of the incident light.

### Example 1

The absorption line of the D1 line of Rb was measured using the vapor cell 10 illustrated in FIGS. 2A and 1B. In the vapor cell 10 used, the reflection space 14 and the storage space 15 are vacuum-sealed. Measurement was performed in a state where the vapor cell 10 was heated to 90° C., and a laser having a wavelength range of 795 nm and a diameter of 200 mm was incident as incident light from VCSEL (vertical cavity surface emitting laser). In the measurement, the current applied to the laser was modulated to change the wavelength. A photodiode was used to detect the emission light. Further, in order to prevent disturbance of a magnetic field, the vapor cell 10 was covered with a permalloy as a magnetic shield.

The measurement result of the absorption line is illustrated in FIG. 7. As illustrated in FIG. 7, each absorption line of the D1 line of Rb was clearly confirmed. The absorption lines outside ±2 GHz in FIG. 7 are the absorption lines of 87Rb, and the absorption lines inside ±2 GHz are the absorption lines of 85Rb.

Subsequently, the incident light was frequency-modulated in the vicinity of the CPT (Coherent Population Trapping) resonance frequency of 3.4 GHz, and the CPT spectrum was measured. The same device as used in the absorption line measurement was used for the measurement, and an electro-optical modulator was used for the intensity modulation of the incident light. The measurement result of the CPT spectrum is illustrated in FIG. 8. As illustrated in FIG. 8, it was confirmed that the peak width of dark resonance was narrow and the frequency shift was small. The half-value width of the peak was 1.40 MHz.

In this way, the vapor cell 10 showed a clear absorption line and had a narrow peak width of the CPT spectrum. Therefore, the vapor cell 10 can be used for a high-precision atomic clock or a high-precision magnetic sensor capable of measuring biomagnetism generated by a heartbeat or an electroencephalogram.

### REFERENCE SIGNS LIST

10: Vapor cell

11: Upper glass plate

12: Silicon wafer

13: Lower glass plate

14: Reflection space

15: Storage space

16: Incident/emission light reflection surface

17: In-plane reflection portion

17a: First reflection surface

17b: Second reflection surface

18: Alkali metal dispenser

19: Dielectric multilayer film

21: SiO₂ film

22, 23: Resist film

24: Stencil mask

16a: Incident light reflection surface

16b: Emission light reflection surface

17c: Third reflection surface

1. A vapor cell comprising:
a reflection space configured to store gas containing an alkali metal atom; and
an incident light reflection surface, an in-plane reflection portion, and an emission light reflection surface provided inside the reflection space, wherein:
the incident light reflection surface has an elevation angle of approximately 45° from an optical path plane so that incident light incident from an external predetermined direction is reflected in an optical path plane that is substantially perpendicular to the incident light,
the in-plane reflection portion has a reflection surface that reflects reflected light from the incident light reflection surface, the reflection surface being substantially perpendicular to the optical path plane so that the reflected light from the incident light reflection surface is reflected in the optical path plane once or multiple times, and
the emission light reflection surface has an elevation angle of approximately 45° from the optical path plane so that reflected light from the in-plane reflection portion is reflected in a direction substantially perpendicular to the optical path plane and an emission light is emitted to the outside.

2-14. (canceled)

15. The vapor cell according to claim 1, wherein the emission light reflection surface is provided so as to emit the emission light in a direction parallel to and opposite to an incident direction of the incident light.

16. The vapor cell according to claim 1, wherein the incident light reflection surface and the emission light reflection surface are formed of the same one surface, and
the in-plane reflection portion is provided so that the reflected light reflected by the incident light reflection surface and the reflected light incident on the emission light reflection surface travel in opposite directions and in parallel to each other.

17. The vapor cell according to claim 16, wherein the in-plane reflection portion has a first reflection surface provided to reflect the reflected light reflected by the incident light reflection surface and bend a traveling direction of the reflected light by 90° and a second reflection surface provided to reflect reflected light reflected by the first reflection surface and bend a traveling direction of the reflected light by 90°.

18. The vapor cell according to claim 1, wherein the incident light reflection surface, the reflection surface of the in-plane reflection portion that reflects the reflected light from the incident light reflection surface, and the emission light reflection surface are covered with a dielectric multilayer film.

19. The vapor cell according to claim 1, wherein the alkali metal atom is Cs or Rb.

20. The vapor cell according to claim 1, wherein the reflection space is sealed.

21. The vapor cell according to claim 1, further comprising a storage space for storing an alkali metal dispenser capable of releasing the alkali metal atom, the storage space being provided such that air can pass between the storage space and the reflection space.

22. A method for manufacturing the vapor cell according to claim 1, comprising:
performing crystal anisotropic etching on a planar silicon to form the incident light reflection surface and the emission light reflection surface; and
performing deep reactive ion etching (DRIE) on the silicon to form the reflection surface of the in-plane reflection portion that reflects reflected light from the incident light reflection surface.

23. The method according to claim 22, wherein the silicon is formed of a silicon wafer having an off angle of 9.74° from the (100) plane.

24. The method according to claim 22, wherein hydrogen annealing is performed at a temperature of 1000° C. or higher after the crystal anisotropic etching and the deep reactive ion etching are performed.

25. The method according to claim 22, wherein after the crystal anisotropic etching and the deep reactive ion etching are performed, a dielectric multilayer film is formed by deposition on the incident light reflection surface, the emission light reflection surface, and the reflection surface of the in-plane reflection portion.

26. The method according to claim 25, wherein the deposition is performed so that a deposition material collides with the incident light reflection surface, the emission light reflection surface, and the reflection surface of the in-plane reflection portion at the same angle.

27. The method according to claim 22, wherein after the incident light reflection surface, the emission light reflection surface, and the reflection surface of the in-plane reflection portion are formed, the silicon is sandwiched between a pair of glass plates to seal the reflection space.

28. The method according to claim 24, wherein after the hydrogen annealing is performed, a dielectric multilayer film is formed by deposition on the incident light reflection surface, the emission light reflection surface, and the reflection surface of the in-plane reflection portion.

29. The method according to claim 25, wherein after the dielectric multilayer film is formed, the silicon is sandwiched between a pair of glass plates to seal the reflection space.
